(19) United States
(12) Patent Application Publication
(10) Pub. No.: US 2023/0152302 A1
(43) Pub. Date: May 18, 2023

(54) SYSTEMS FOR DETECTION

(71) Applicant: KONIKU INC., San Rafael, CA (US)

(72) Inventors: Jean-Charles Neel, San Francisco, CA (US); Michael Siani-Rose, San Francisco, CA (US); Oshiorenoya Agabi, Dublin, CA (US)

(21) Appl. No.: 18/151,345

(22) Filed: Jan. 6, 2023

Related U.S. Application Data
(63) Continuation of application No. 16/344,791, filed on Apr. 24, 2019.
(60) Provisional application No. 62/413,897, filed on Oct. 27, 2016.

(51) Int. Cl. G01N 33/50; G01N 33/543; G01N 33/94

(52) U.S. Cl. CPC G01N 33/5058; G01N 33/5438; G01N 33/94

(57) ABSTRACT

Devices, systems, and methods detect compounds, such as odorant compounds. Patterns of electrical signals are detected with a given pattern associated with a given compound or a mixture of compounds such that a presence of the given compound or mixture of compounds can be determined.

### CROSS-REFERENCE TO RELATED APPLICATION(S)

This application is a continuation of U.S. patent application Ser. No. 16/344,791 filed Apr. 24, 2019 and now pending, which claims priority to U.S. Provisional application 62/413,897, filed on Oct. 27, 2016. These applications are incorporated herein by reference.

### BACKGROUND

Cellular arrays are of utility in medical research and life sciences in general. Traditional cellular arrays use simple containers such as a petri dish or multi-well plate as a vessel for cell culture. However, it is recognized in the art that such a simple approach provides cells with a substantially different environment to that experienced by cells in vivo.

### SUMMARY

The system may comprise an array, at least one electrode positioned within each chamber of the plurality of chambers to form a plurality of electrodes configured to measure electrical signals; and a controller configured to receive the electrical signals measured by the plurality of electrodes. In some embodiments, the array may comprise a plurality of chambers, wherein each of the plurality of chambers may comprise a cell expressing one or more cell- surface receptors. In some embodiments, when a compound is introduced into a chamber of the plurality of chambers and a binding event occurs between one or more of the cell-surface receptors of a cell and the compound, an electrical signal may result in response to the binding. In some embodiments, the controller may generate a pattern of electrical signals associated with the compound.

At least one electrode may be configured to measure an electrical signal of a cell housed within a respective chamber. In some embodiments, two or more compounds may be introduced. In some embodiments, the compound may be a volatile compound.

The pattern of electrical signals may be a compound- specific pattern. In some embodiments, the compound-specific pattern may provide a confirmation of a presence of the compound that is introduced into the chamber. The pattern of electrical signals may be specific to a collection of compounds. In some embodiments, the pattern of electrical signals may provide a unique fingerprint for identifying a presence of the compound in a sample. In some embodiments, the system may be an odorant- detection system.

The one or more cell-surface receptors may be odorant receptors. In some embodiments, the cell may be a neuron. The electrical signals may comprise an action potential. In some embodiments, the electrical signals may comprise an excited signal level that is below a threshold for an action potential. The electrical signals may comprise a cell membrane depolarization.

The pattern of electrical signals may comprise a binary pattern. In some embodiments, the pattern of electrical signals may comprise magnitudes of individual electrical signals received from individual electrodes. The pattern of electrical signals may comprise temporal patterns of electrical signals received from individual electrodes. In some embodiments, the controller may receive the pattern of electrical signals and forms a matrix based on the pattern received. The controller may store patterns of electrical signals associated with specific compounds in a database.

The cell may be modified to express one or more cell- surface receptors. In some embodiments, the cell may be genetically modified to express one or more cell-surface receptors. The cell-surface receptors may be modified cell-surface receptors. The modified cell-surface receptors may comprise a genetic modification, a methylation modification, a sulfonation modification, a sulfentation modification, an acylation modification, an alkylation modification, a butyrylation modification, a glycosylation modification, a malonylation modification, a hydroxylation modification, an iodination modification, a propionylation modification, or any combination thereof.

The pattern of electrical signals may represent a probability of a presence of the compound. In some embodiments, the probability may be at least about 75%. In some embodiments, each chamber of the plurality of chambers may be operatively coupled to a respective cell introduction port by a respective cell introduction passage. The system further may comprise at least one perfusion channel fluidically coupled to a chamber of the plurality of chambers.

Another aspect provides a method for detecting a presence or a likelihood of a presence of a compound. In some embodiments, the method may detect a presence of a neurotoxin, a toxin, a volatilized plant component, or any combination thereof. In some embodiments, the volatilized plant component may comprise tobacco, marijuana, tetrahydrocannabinol (THC), nicotine, cocaine, or any combination thereof In some

The method may detect a presence of an illegal substance as defined in 42 United States Code § 12210, a carcinogen or a chemical weapon, which may be a mustard gas, a sarin gas, or any combination thereof. In some embodiments, the method may detect a presence of a thyomethane, a hydrocarbon, an oxygen, a carbon dioxide, or any combination thereof

The method may provide for confirming a presence or absence of a compound in the sample. The method may comprise (a) adding a sample to at least one of the plurality of chambers; (b) measuring one or more electrical signals employing the plurality of electrodes; (c) generating a pattern of electrical signals; (d) comparing the pattern of electrical signals to one or more compound- specific patterns stored in a database of the system; and (e) confirming a presence or absence of a compound in the sample based on the comparing.

In one aspect, a device comprises: (a) a spatially addressable array, the spatially addressable array comprising: a plurality of chambers, wherein each chamber of the plurality of chambers comprises: (i) a cell modified to express a unique odorant receptor profile; and (ii) an electrical component configured to measure an electrical signal in the cell, (b) a controller configured to (i) receive the measured electrical signals from the plurality of chambers and (ii) determining a presence or an absence of one or more compounds based on the measured electrical signals.

In another aspect, a method of detecting a presence or an absence of one or more compounds in an environment, the method comprising: (a) placing a spatially addressable array in the environment, wherein the spatially addressable array comprises: a plurality of chambers, wherein each chamber of the plurality of chambers comprises: (i) a cell modified to express a unique odorant receptor profile; and (ii) an electrical component configured to measure an electrical signal in the cell; and (b) detecting the presence or the absence of the one or more compounds based the measured electrical signals from the plurality of chambers.

In another aspect, provided herein is a device comprising: an array, the array comprising a plurality of chambers, wherein each of the plurality of chambers comprises: (i) a cell modified to express a cell receptor having a binding specificity for a compound selected from the group consisting of a neurotoxin, a carcinogen, a chemical weapon, and any combination thereof; and (ii) an electrical component configured to measure one or more signals, wherein the device is configured to detect a presence or an absence of the compound based on one or more signals measured in the array.

In another aspect, provided herein is a method of detecting a presence or an absence of a compound in an environment, the method comprising: (a) placing a device in the environment, wherein the device comprises: an array, the array comprising a plurality of chambers, wherein each of the plurality of chambers comprises: (i) a cell modified to express a cell-surface receptor having a binding specificity for the compound, wherein the compound is selected from the group consisting of a neurotoxin, a carcinogen, a chemical weapon, and any combination thereof; and (ii) an electrical component configured to measure one or more signals; and (b) detecting the presence or the absence of the compound based on one or more signals measured in the device.

In another aspect, provided herein is an array of n different cells, each of which expresses a unique odorant receptor, wherein the array is capable of detecting greater than different compounds each at a confidence level greater than about 70%.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an array of cells on a micro- electrode array (MEA).

FIG. 2 shows a neuron expressing an odorant receptor surrounding an electrode.

FIG. 3 shows a computer control system that is programmed or otherwise configured to implement methods provided herein.

FIG. 4 shows a schematic view of a micro- electrode array (MEA) comprising an array of cells.

FIG. 5 provides a table of compounds matched with odorant receptors and the concentration limit of detection for each compound using the respective odorant receptor to detect.

FIG. 6 shows a range of DNT (CAS #121-14-2) concentrations (1 microMolar (uM) to 1000 (uM)) and respective odorant receptor detection as reported by measured levels of luminescence.

FIG. 7 shows a range of vanillic acid (CAS#121-34-6) concentrations (100 picoMolar (pM) to 1 milliMolar (mM)) and respective odorant receptor detection as reported by measured levels of luminescence.

FIG. 8 shows a range of DNT (CAS #121-14-2) concentrations (100 picoMolar (pM) to 1 milliMolar (mM)) and respective odorant receptor detection as reported by measured levels of luminescence.

FIG. 9 shows an action potential of a neuron recorded using a planar electrode.

FIG. 10 shows an action potential of a neuron recorded using a 3D electrode.

FIG. 11 show a spike train obtained using a 3D electrode.

FIG. 12 shows odorant receptor detection of four different compounds (cocaine, heroin, LSD, and PCP) as measured levels of luminescence (y-axis) for a panel of different odorant receptors types (x-axis).

FIG. 13 shows odorant receptor detection of vanillic acid as measured by levels of luminescence (y-axis) at different concentrations (x-axis) for six different types of odorant receptors (legend).

FIG. 14 shows odorant receptor detection as measured by levels of luminescence (y-axis) for a panel of different types of compounds (x-axis) for mouse odorant receptor (mOR9-I).

FIG. 15 shows an electrical signal comprising a spike burst from a broadly tuned receptor for a high affinity compound.

FIG. 16 shows an electrical signal comprising a spike burst from a broadly tuned receptor for a low affinity compound.

FIG. 17 shows a raster plot of Gaussian noise or baseline noise of the detection device.

FIG. 18 shows a raster plot showing spike bursts for two different narrowly tuned receptors. The first narrowly tuned receptor detects odorant A at 500-100 time and the second narrowly tuned receptor detects odorant B at 1500-2000 time. The x-axis is time. The y-axis is cell number.

FIG. 19 shows a raster plot showing spike bursts for broadly tuned receptors. The x-axis is time. The y-axis is cell number.

FIG. 20 shows a raster plot showing spike bursts for broadly tuned receptors. The x-axis is time. The y-axis is cell number.

FIG. 21 shows results for narrowly tuned receptors with electrical signals directly recorded from the neurons having the narrowly tuned receptors. The top panel shows the raw data in a raster plot. The middle panel shows the experimental conditions (or experimental truth) of time exposure for two different compounds (indicated as B or R). The bottom panel shows the predicted outcome.

FIG. 22 shows results for broadly tuned receptors with electrical signals directly recorded from the neurons having the broadly tuned receptors. The top panel shows the raw data in a raster plot. The middle panel shows the experimental condition (or experimental truth) of the time exposure for two different compounds (indicated as B or R). The bottom panel shows the predicted outcome.

FIG. 23 shows results for broadly tuned receptors with electrical signals recorded from another neuron in communication with the primary neuron having the broadly tuned receptors. The top panel shows the raw data in a raster plot. The middle panel shows the experimental condition (or experimental truth) of the time exposure for two different compounds (indicated as B or R). The bottom panel shows the predicted outcome.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a”, “an”, and “the” include plural references unless the context clearly dictates otherwise. Any reference to “or” herein is intended to encompass “and/or” unless otherwise stated.

As used herein, the term “about” means the referenced numeric indication plus or minus 15% of that referenced numeric indication.

The term “cell” as used herein, generally refers to one or more cells. A cell may be obtained or isolated from a subject. A cell may be obtained or isolated from a tissue. A subject may be an animal such as a human, a mouse, a rat, a pig, a dog, a rabbit, a sheep, a horse, a chicken or other. A cell may be a neuron. A neuron may be a central neuron, a peripheral neuron, a sensory neuron, an interneuron, a motor neuron, a multipolar neuron, a bipolar neuron, or a pseudo-unipolar neuron. A cell may be a neuron supporting cell, such as a Schwann cell. A cell may be one of the cells of a blood-brain barrier system. A cell may be a cell line, such as a neuronal cell line. A cell may be a primary cell, such as cells obtained from a brain of a subject. A cell may be a population of cells that may be isolated from a subject, such as a tissue biopsy, a cytology specimen, a blood sample, a fine needle aspirate (FNA) sample, or any combination thereof A cell may be obtained from a bodily fluid such as urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid. A cell may comprise cancerous cells, non- cancerous cells, tumor cells, non- tumor cells, healthy cells, or any combination thereof A cell may be a modified cell, such as a genetically modified cell. A modified cell may comprise an addition of one of more cell-surface receptors, such as modified cell-surface receptors. The modified cell-surface receptors may be modified to increase or decrease their ability to bind to a large set of compounds, a small set of compounds, or a specific compound. A modified cell may comprise a deletion of one or more cell-surface receptors.

The term “tissue” as used herein, generally refers to any tissue sample. A tissue may be a sample suspected or confirmed of having a disease or condition. A tissue may be a sample that is genetically modified. A tissue may be a sample that is healthy, benign, or otherwise free of a disease. A tissue may be a sample removed from a subject, such as a tissue biopsy, a tissue resection, an aspirate (such as a fine needle aspirate), a tissue washing, a cytology specimen, a bodily fluid, or any combination thereof. A tissue may comprise cancerous cells, tumor cells, non-cancerous cells, or a combination thereof. A tissue may comprise neurons. A tissue may comprise brain tissue, spinal tissue, or a combination thereof. A tissue may comprise cells representative of a blood-brain barrier. A tissue may comprise a breast tissue, bladder tissue, kidney tissue, liver tissue, colon tissue, thyroid tissue, cervical tissue, prostate tissue, lung tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof.

The term “receptor” as used herein, generally refers to a receptor of a cell. The receptor may be a cell-surface receptor. A cell-surface receptor may be a G coupled protein receptor. A receptor may bind to one or more compounds. A receptor may have a different binding affinity to for each compound to which it binds. A receptor may be modified, such as genetically modified. A receptor may be modified to change the number of compounds to which it may bind. A receptor may be modified to increase the number of different compounds to which it may bind. A receptor may be modified to decrease the number of different compounds to which it may bind. A receptor may bind 1 compound. A receptor may bind 2, 3, 4, 5, 6, 7, 8, 9, 10, 15, 20, 30 ,40, 50, 60, 70, 80, 90, 100 compounds or more. A receptor may bind less than 10 compounds. A receptor may bind less than 5 compounds. A receptor may bind at least 5 compounds. A receptor may bind at least 10 compounds. A receptor may bind at least 20 compounds.

The term “modification” as used herein, generally refers to a modification to a cell or cell receptor. A modification to a cell may include adding one or more receptors, such as modified receptors, to the cell. A modification to a cell may include removing one or more receptors from a cell. A modification to a cell may include modifying one or more receptors that are expressed on the cell. A modification to a cell receptor may include a genetic modification.

The term “compound” as used herein, generally refers to a composition that may produce a signal in a cell, such as an electrical signal. A compound may comprise an odorant. A compound may comprise a compound that binds an odorant receptor or a modified odorant receptor. A compound may comprise a volatile compound or an organic volatile compound. A compound may comprise a neurotoxin or a toxin; a carcinogen; a chemical weapon, such as a mustard gas, a sarin gas, or a combination thereof. A compound may comprise an illegal substance as defined in 42 United States Code § 12210. A compound may comprise a drug or a pharmaceutical composition or salt thereof A compound may comprise a protein, a peptide, a nucleic acid, an antibody, an aptamer, a small molecule. A compound may comprise a cell or a cellular fragment. A compound may comprise a tissue or tissue fragment. A compound may comprise a naturally-derived composition or a synthetic composition. A compound may be an explosive compound, such as trinitrotoluene (TNT). A compound may be a precursor to the compound (such as a chemical precursor), a degradation product of the compound, or a metabolite of the compound, or any combination thereof A compound may be any compound described herein, including DNT, RDX, TNT, vanillic acid, or others.

The term “sample” as used herein, generally refers to a sample that may or may not comprise one or more compounds. A sample may be tissue or fluid sample obtained from a subject, such as a human subject. A sample may be a fluid or gas sample obtained from an air space, such as an air space adjacent to a deployment of a chemical weapon or an air space in a residential or commercial setting. A sample may be a blood sample obtained from a subject. A sample may be a soil sample, such as a sample obtained near a tracking system or oil rig system. A sample may be a sample that may comprise a compound that is an environmental hazard or a health hazard. A sample may be a liquid sample obtained from a water system, such as a river, a stream, a lake, an ocean, or others. A sample may be a food sample or a container system that houses a food sample. A pattern or fingerprint of the systems described herein, may confirm a ripeness of a single piece of food, such as a fruit, or a set of fruit.

The term “signal” as used herein, generally refers to a signal in response to a binding event, for example, a compound binding to a cell- surface receptor of a cell. The signal may be an electrical signal. The signal may be a change in a cell membrane potential. The signal may be a membrane depolarization. The signal may be an action potential. The signal may be an electrical signal that is subthreshold of an action potential. The signal may be a magnitude of a change in a cell membrane potential, or a magnitude of an action potential. The signal may be the number of action potentials or a train of action potentials. The signal may be a signal measured over a period of time. Information from a signal may be imported into a matrix to form a fingerprint or a pattern of signals. The fingerprint or pattern of signals may be a unique fingerprint. The signal may be a measurement of an amplitude, a period, or a frequency, of a combination thereof of an electrical signal. The signal may be a time length of a refractory period following an action potential. The signal may be a peak voltage of an action potential. The signal may be a time to a peak voltage of an action potential. The signal may be a peak voltage of a membrane depolarization.

The term “surface roughness” as used herein, generally refers to surface texture or to an amplitude and/or a frequency of deviations on a surface. The deviations may be protrusions and/or recesses. The deviations may form a regular pattern or may be random.

Sensitivity may refer to TP/(TP+FN), where TP may be true positive (correctly detecting a presence of a compound in an environment or sample) and FN may be false negative (incorrectly detecting an absence of a compound in an environment or sample). An array may detect a presence or an absence of one or more compounds at a sensitivity of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds. In some cases, increasing the number of unique odorant receptors within an array may increase the sensitivity of detection for one or more compounds.

Specificity may refer to TN/(TN+FP), where TN may be true negative (correctly detecting an absence of a compound in an environment or sample) and FP may be false positive (incorrectly detecting a presence of a compound in an environment or sample). An array may detect a presence or an absence of one or more compounds at a specificity of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds. Increasing the number of unique odorant receptors within an array may increase the specificity of detection for one or more compounds.

Positive Predictive Value (PPV) may refer to TP/(TP+FP). A PPV may be the proportion of samples with positive test results that correctly detect a presence or an absence of a compound. An array may detect a presence or an absence of one or more compounds at a PPV of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

Negative Predictive Value (NPV) may refer to TN/(TN+FN). An array may detect a presence or an absence of one or more compounds at an NPV of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

An array as described herein may detect a presence or an absence of one or more compounds at an accuracy of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

An array as described herein may detect a presence or an absence of one or more compounds at a confidence level of greater than about: 70%, 75%, 80%, 85%, 86%, 87%, 88%, 89%, 90%, 91%, 92%, 93%, 94%, 95%, 96%, 97%, 98%, 99%, or 99.5% tor the one or more compounds.

An array as described herein may detect a presence or an absence of one or more compounds at one or more of a sensitivity, a specificity, a PPV, an NPV, an accuracy, a confidence level, or any combination thereof at greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

A device as described herein may be placed into an environment. The environment may be a residential setting, such as an indoor or outdoor residential setting. The environment may be a public space. The public space may include an internal public environment such as a public building or may include a public outside space. The environment may be a privately owned space such as a privately owned indoor space (such as a building) or a privately owned outdoor space. A device as described herein may be configured to receive a sample collected from an environment, such as a residential setting or a public space. A sample obtained from an environment may be added to at least a portion of the device. A sample from an environment may contact at least a portion of the device when the device may be placed into the environment.

An array may comprise a unique receptor, such as an odorant receptor. An array may comprise two or more unique receptor profiles. An array may comprise three or more unique receptor profiles. An array may comprise four or more unique receptor profiles. An array may comprise five or more unique receptor profiles. An array may comprise six or more unique receptor profiles. An array may comprise 2, 3, 4, 5, 6, 7, 8, 9, 10, 11, 12, 13, 14, 15, 16, 17, 18, 19, 20 or more unique receptor profiles. An array may comprise from 1-10 unique receptor profiles. An array may comprise from 1-20 unique receptor profiles. An array may comprise from 1-50 unique receptor profiles. An array may comprise from 1-100 unique receptor profiles. An array may comprise from 5-20 unique receptor profiles. A unique receptor may be an odorant receptor. A unique receptor may be a mouse receptor. A unique receptor may be a human receptor. A unique receptor may be an insect receptor.

A device may be configured to detect a presence or an absence of a compound. A device may be configured to detect a presence or an absence of a subset of structurally related compounds. A device may be configured to detect a presence or an absence of a subset of compounds related by function or use (such as explosive compounds or drug compounds). Detection for each compound of a subset may include a confidence level, accuracy, sensitivity, specificity, PPV, NPV or a combination thereof greater that about: 80%, 85%, 90%, 95%, 99% or more.

A device may be configured to detect a presence or an absence of a panel of unique compounds. A device may be configured to detect a presence or an absence of at least two unique compounds. A device may be configured to detect a presence or an absence of at least three unique compounds. A device may be configured to detect a presence or an absence of at least four unique compounds. A device may be configured to detect a presence or an absence of at least five unique compounds. A device may be configured to detect a presence or an absence of at least six unique compounds. A device may be configured to detect a presence or an absence of at least 2, 3, 4, 5, 6, 7, 8, 9, 10, 11, 12, 13, 14, 15, 16, 17, 18, 19, 20 or more unique compounds. A device may be configured to detect a presence or an absence of from 1-10 unique compounds. A device may be configured to detect a presence or an absence of from 1-20 unique compounds. A device may be configured to detect a presence or an absence of from 1-50 unique compounds. A device may be configured to detect a presence or an absence of from 1-100 unique compounds. A device may be configured to detect a presence or an absence of from 5-20 unique compounds.

A cell may be capable of detecting a presence or an absence of more than one unique compound. For example, a cell may comprise a receptor that may be capable of detecting more than one unique compound, such as a broadly tuned receptor. In another example, a cell may comprise two unique receptors, each of which may be capable of detecting a unique compound, such as two narrowly tuned receptors for each of the unique compounds being detected. A cell may be capable of detecting a presence or an absence of 2, 3, 4, 5, 6, 7, 8, 9, 10, 11, 12, 13, 14, 15, 16, 17, 18, 19, 20 or more unique compounds. A cell may be capable of detecting a presence or an absence of from 1-10 unique compounds. A cell may be capable of detecting a presence or an absence of from 1-20 unique compounds. A cell may be capable of detecting a presence or an absence of from 1-50 unique compounds. A cell may be capable of detecting a presence or an absence of from 1-100 unique compounds. A cell may be capable of detecting a presence or an absence of from 5-20 unique compounds.

A cell may be modified to express a receptor. The receptor may be an odorant receptor. The receptor may be a wild-type receptor. The receptor may be a modified receptor, such as a genetically modified receptor. A receptor may be modified to enhance a binding specificity to a particular compound or to alter the receptor from a broadly tuned receptor to a narrowly tuned receptor or vice versus. The cell may be modified to express more than one unique receptor. The cell may be modified to express two unique receptors. The cell may be modified to express three or more unique receptors. A receptor may be a human receptor, a mouse receptor, an insect receptor, or other species type of odorant receptor.

A device may comprise one or more unique receptor profiles. For example, a device may comprise 1, 2, 3, 4, 5, 6, 7, 8, 9, 10, 15, 20, 50 or more unique receptor profiles. A device may comprise receptors that may be broadly tuned to a particular compound and receptors that may be narrowly tuned to a particular compound. For example, a broadly tuned receptor may bind to a particular compound with a lower specificity than a narrowly tuned receptor. For example, a broadly tuned receptor may bind to a greater number of off-target compounds in addition to the particular compound of interest as compared to the lower number of off-target compounds that bind to a narrowly tuned receptor. A device may comprise a panel of receptors, each receptor having a different tuning for a particular compound.

A benefit of incorporating broad and narrow tuned receptors into an array may be (i) an increased specificity, an increased sensitivity, an increased confidence level, an increased accuracy, an increased PPV or NPV for diagnosing a presence or an absence of a particular compound in an environment, (ii) a lower concentration threshold for detection of one or more compounds, or (iii) a combination thereof An electrical signal obtained from cell having a narrowly tuned receptor may be unique and distinguishable from an electrical signal obtained from a cell having a broadly tuned receptor. An array having a combination of broadly tuned and narrowly tuned receptors for a given compound may provide a unique fingerprint of detection (comprising measured electrical signals) for the presence or the absence of the compound in a sample or environment. A unique fingerprint for a given compound may be a signaling pattern. A unique fingerprint for a given compound may comprise (i) a spatial pattern of chambers of the array having measured patterns of electrical signals, (ii) a pattern of electrical signals measured within a chamber of the array, or (iii) a combination thereof. A unique fingerprint for a given compound may also be specific to the combination of receptor profiles of the array.

### Electrical Components

An electrical component may comprise an electrode. An electrode may be a two-dimensional electrode. An electrode may be a three-dimensional electrode. An electrical component may comprise one or more sensors, such as a temperature sensor, a pH sensor, a gas sensor, a glucose sensor, a level sensor, or any combination thereof In some embodiments, the gas sensor may be an 0₂ sensor, a C0₂ sensor, or a combination thereof In some embodiments, the one or more sensors may comprise an optical sensor, an electrochemical sensor, an opto-electric sensor, a piezoelectric sensor, a biosensor, or any combination thereof In some embodiments, each chamber of the plurality of chambers may comprise at least one sensor. In some embodiments, the device may further comprise a controller configured to instruct the electrical components to collect one or more measurements respective to the one or more sensors.

An electrode may comprise a metal. An electrode may comprise an alloy. An electrode may comprise aluminum, gold, lithium, copper, graphite, carbon, titanium, brass, silver, platinum, palladium, cesium carbonate, molybdenum(VI) oxide, or any combination thereof An electrode may comprise a mixed metal oxide.

Modifying an electrode with a plurality of protrusions, a plurality of recesses, modifying by adding a surface roughness to the surface of an electrode may increase the surface area of the electrode. This modification or enhanced surface area may enhance the amount of cellular attachment to the electrode. This modification may enhance the portion of the electrode that is contacted or at least partially engulfed by a cell. This modification may enhance the portion of the electrode that is contacted by a cell. This modification may enhance an electrical connection between a cell and an electrode.

An electrode may comprise a spherical shape, a hemispherical shape, a mushroom shape, comprising a head portion and support portion, a rod-like shape, a cylindrical shape, a conical shape, a patch shape, or any combination thereof A cell culture module may comprise electrodes having the same shape. For example, a module may comprise 10 electrodes of a mushroom shape. A cell culture module may comprise electrodes of more than one type of shape. For example, a module may comprise 10 electrodes of a mushroom shape and 10 electrodes of a conical shape.

An electrode may have a combination of one or more protrusions and one or more recesses. An electrode may have a combination of one or more protrusion shapes, such as a hemispherical protrusion, and one or more recess shapes, such as a hemispherical recess.

A protrusion may be a hemispherical protrusion. A protrusion may be a spike protrusion, a conical protrusion, a square or rectangular rod protrusion, an obelisk protrusion, a cylindrical protrusion, a hemispherical protrusion, or any combination thereof A recess may be a hemispherical recess. A recess may be a V-groove recess, a dovetail recess, a spike recess, a conical recess, a cylindrical recess, a square or rectangular rod recess, a hemispherical recess, or any combination thereof. An electrode may have one or more protrusions. An electrode may have a surface concentration of protrusions of about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, 10 protrusions per micrometer squared (pro/um²). An electrode may have a surface concentration of protrusions of 0.0001 to about 0.01 pro/um². An electrode may have one or more recesses, and a surface concentration of recesses from about 0.0001 to about 0.01 rec/um².

The surface of an electrode may be smooth. The surface of an electrode may have a surface roughness. A surface roughness may be uniform across the surface of an electrode. A portion of the surface of an electrode may have a surface roughness, such as a top portion of the electrode, a bottom portion of the electrode. An electrode may have alternating rows of smooth and rough portions. A surface roughness may be from about 5 to about 50 nm.

A width of an electrode may be of a size to accommodate a cell to contact or at least partially engulf the electrode. A width of an electrode may be about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, 25, 26, 27, 28, 29, 30, 35, 40, 45, 50 micrometers (um). A width of an electrode may be a width of the support portion. A width of an electrode may be a width of the head portion.

A medium may include one or more components. For example, a medium may include one or more pH modulating agents. The one or more pH modulating agents may include a buffering agent, an inorganic salt, or any combination thereof. An inorganic salt may include sodium phosphate dibasic, sodium phosphate monobasic, or any combination thereof.

A medium may include one or more components. For example, a medium may include one or more supplemental agents. The one or more supplemental agents may include a protein, a neurotrophic factor, a steroid, a hormone, a fatty acid, a lipid, a vitamin, a sulfate mineral, an organic chemical compound, a monosaccharide, a nucleotide, or any combination thereof.

A medium may include one or more components. For example, a medium may include one or more energetic substrates. The one or more energetic substrates may include a sugar, sodium pyruvate, or a combination thereof. The energetic substrate may be present in the medium at a concentration of between about 0.1 and about 5 mM. The energetic substrate may be present in the medium at a concentration of between about 5 and about 10 mM. The energetic substrate may be present in the medium at a concentration of between about 0.001 and about 0.09 mM. The energetic substrate may be present in the medium at a concentration of between about 1 and about 5 mM. The energetic substrate may be present in the medium at a concentration of between about 0.1 and about 1 mM.

A medium may include one or more components. For example, a medium may include one or more light sensitive agents. The light sensitive agent may include riboflavin (B2), HEPES, or a combination thereof. The light sensitive agent (such as riboflavin (B2)) may be present in the medium at a concentration of between about 0.0001 and about 0.0006 mM. The light sensitive agent (such as riboflavin (B2)) may be present in the medium at a concentration of between about 0.000001 and about 0.00009 mM. The light sensitive agent (such as riboflavin (B2)) may be present in the medium at a concentration of between about 0.0007 and about 0.06 mM. The light sensitive agent (such as HEPES) may be present in the medium at a concentration of between about 1 and about 10 mM. The light sensitive agent (such as HEPES) may be present in the medium at a concentration of between about 0.001 and about 0.9 mM. The light sensitive agent (such as HEPES) may be present in the medium at a concentration of between about 11 and about 20 mM.

A medium when contacted with an isolated cell may maintain one or more in vivo- like functions of the isolated cell in an ex vivo environment. For example, a medium may maintain an isolated neuron in a vivo-like neurophysiological function by preserving one or more of synaptic function, action potential generation, energetic maintenance, or any combination thereof. An isolated cell cultured in an ex vivo environment may include an environment that is not an in vivo environment. An ex vivo environment may include an environment that does not include one or more incubator conditions (from about 3% to about 8% C02 content; from about 34 degrees C. to about 40 degrees C.; and from about 75% to about 85% humidity). An ex vivo environment may include an environment lacking one or more incubator conditions.

A medium may include one or more components, such as one or more buffering agents. A buffering agent may increase the life span of an isolated cell as compared to a cell cultured in a medium without the buffering agent. A buffering agent added to a medium may increase the life span of an isolated cell cultured outside of incubator conditions (from about 3% to about 8% C02 content; from about 34 degrees C. to about 40 degrees C.; and from about 75% to about 85% humidity) as compared to a cell cultured in a medium without the buffering agent also outside of incubator conditions. A buffering agent may increase the life span of the isolated cell by about: 1 day, 1 week, 2 weeks, 3 weeks, 1 month, 2 months, 3 months, 4 months, 5 months, 6 months, 7 months, 8 months, 9 months, 10 months, 11 months, 12 months or more. A buffering agent may increase the life span of the isolated cell by about from 1 week to 1 month. A buffering agent may increase the life span of the isolated cell by about from 1 month to 3 months. A buffering agent may increase the life span of the isolated cell by about from 1 month to 1 year. A buffering agent may increase the life span of the isolated cell by about from 1 month to 6 months.

### Receptors and Compound Detection

A cell receptor may detect a presence of a compound, such as when a compound binds to the cell receptor. A binding event between a cell receptor and a compound may result in a signal, such as a light signal or electrical signal or chemical signal.

A cell receptor that may detect a presence of a compound may be a wild type receptor. A cell receptor that may detect a presence of a compound may be a modified receptor, such as a genetically modified receptor. A cell receptor that may detect a present of a compound may be an odorant receptor.

A cell, such as a neuron, may be modified to include one or more wild type receptors, one or more modified receptors, or any combination thereof A cell may be modified to include a receptor that detects a single compound. A cell may be modified to include a receptor that detects more than one compound. A cell may be modified to include more than one type of receptor, such as a first type of receptor that detects a first compound and a second type of receptor that detects a second compound. A cell may be modified to include 1, 2, 3, 4, 5, 6, 7, 8, 9 or 10 different types of receptors.

A binding event between a cell receptor and a compound may occur in a liquid medium. A binding event may occur in a semi- solid medium. A binding event may occur in a viscous medium. A binding event may occur in an aqueous medium. A binding event may occur in a hydrogel.

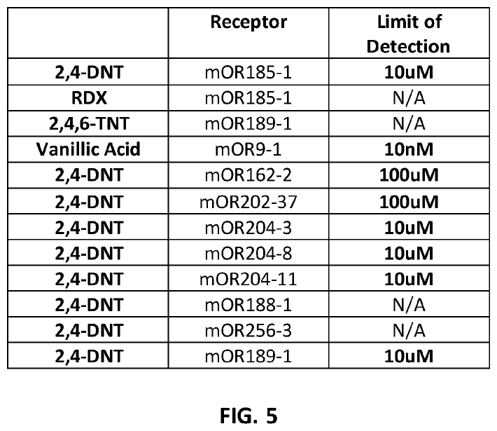

FIG. 5 shows a table of compounds (left column) and receptors that detect such compounds (middle column) and the concentration limit of detection for each compound receptor pair (right column). For example, 2,4-DNT (CAS #121-14-2) may be detected by receptor mOR185-I at a concentration limit of detection of about 10 uM. A single receptor may detect more than one compound. Multiple receptors may detect the same compound at different concentration limits of detection.

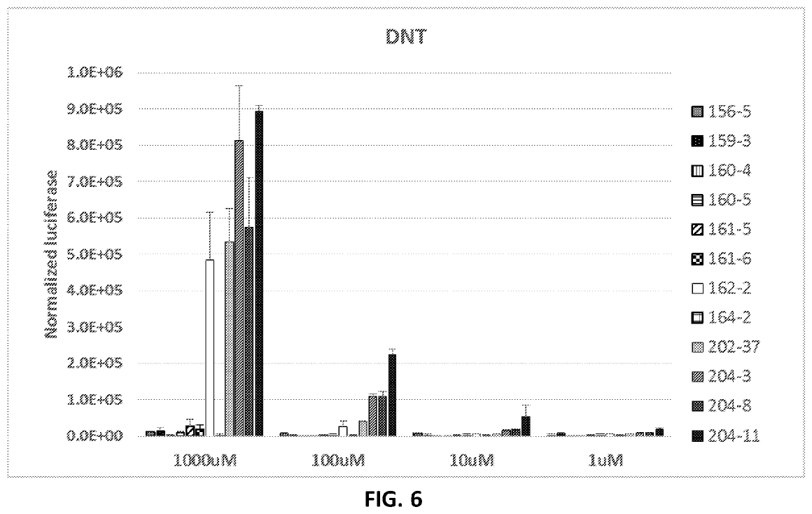

FIG. 6 shows a range of DNT (CAS #121-14-2) concentrations (1 microMolar (uM) to 1000 (uM)) and respective odorant receptor detection as reported by measured levels of luminescence. The y axis of the bar graph reports measured normalized luciferase activity. The x axis of the bar graph reports different concentration limits of detection. The right hand side of the graph reports different receptors tested. Some receptors show a lower limit of detection of DNT than other receptors.

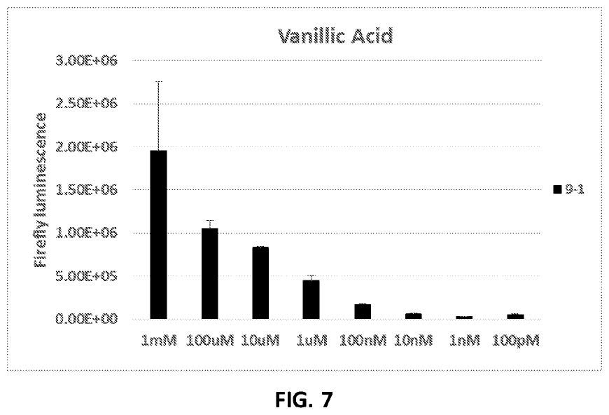

FIG. 7 shows a range of vanillic acid (CAS#121-34-6) concentrations (100 picoMolar (pM) to 1 milliMolar (mM)) and respective odorant receptor detection as reported by measured levels of luminescence. The y axis of the bar graph reports measured luminescence. The x axis of the bar graph reports different concentration limits of detection. The right hand side of the graph reports the receptor mOR9-I tested. A lower limit of detection for vanillic acid by receptor mOR9-I may be about 10 nM.

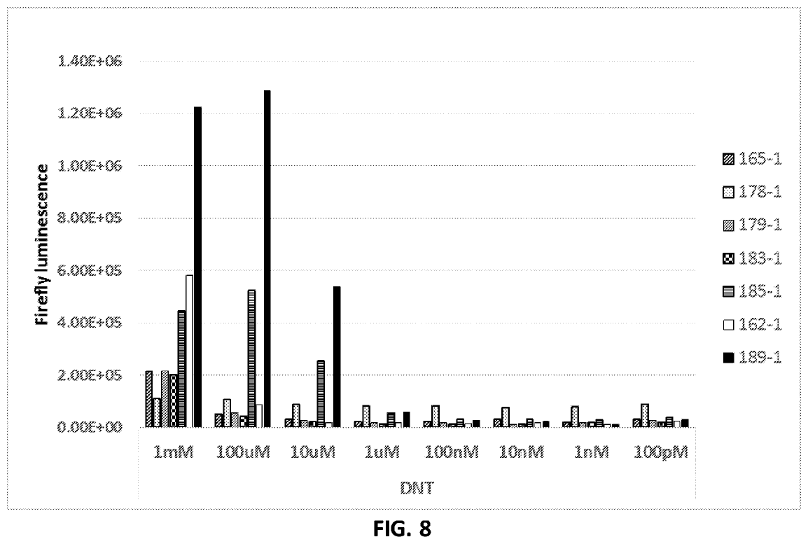

FIG. 8 shows a range of DNT (CAS #121-14-2) concentrations (100 picoMolar)(pM) to 1 milliMolar (mM)) and respective odorant receptor detection as reported by measured levels of luminescence. The y axis of the bar graph reports measured luminescence. The x axis of the bar graph reports different concentration limits of detection. The right hand side of the graph reports different receptors tested. Some receptors show a lower limit of detection of DNT than other receptors.

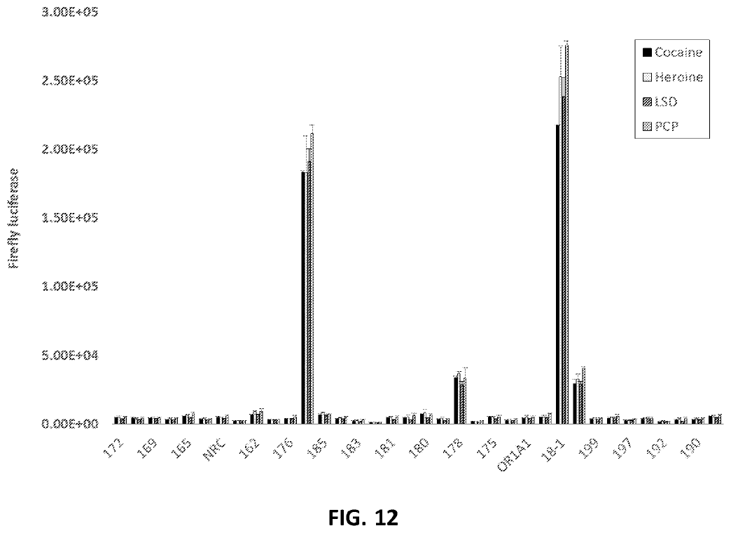

FIG. 12 shows odorant receptor detection of four different compounds: cocaine, heroin, LSD, and PCP as measured by levels of luminescence for a panel of different odorant receptor types. A subset of odorant receptors tested (such as mOR18-2, mOR178-I, mOR18-I and OR2W1) responded to all four compounds tested as indicated by the measured levels of luminescence. Broadly tuned receptor, such as those receptors that detect more than one type of compound, may help detect a subset of compounds without being able to identify the chemical nature of the compound being detected.

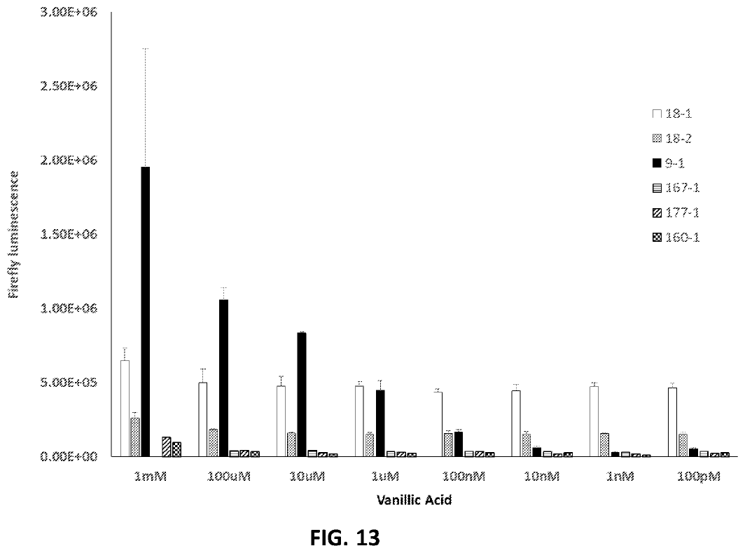

FIG. 13 shows odorant receptor detection of vanillic acid as measured by levels of luminescence at different concentrations for six different types of odorant receptors. Odorant receptor mOR9-1 demonstrates a decreasing level of luminescence as the concentration of vanillic acid is decreased. Odorant receptors mOR18-I and mOR18-2 demonstrate similar levels of luminescence as the concentration of vanillic acid is decreased. These receptors may respond to other environmental stimuli. Odorant receptors mOR177-I and mOR160-I show low background signal in the presence of different concentrations of vanillic acid.

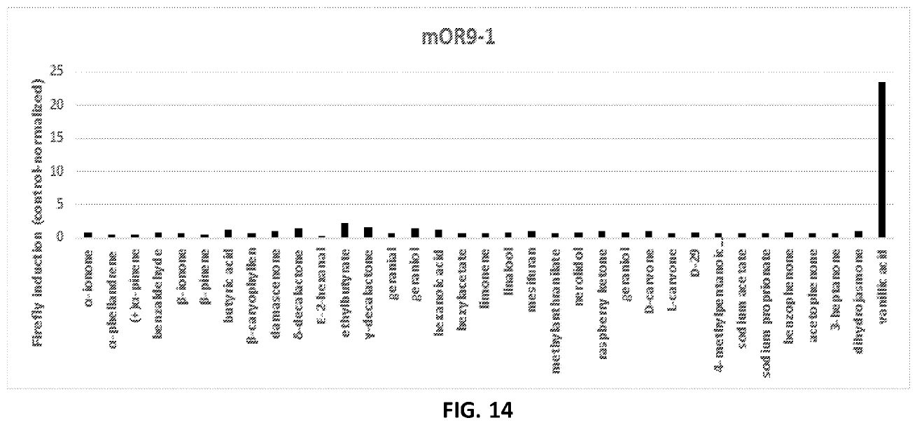

FIG. 14 shows odorant receptor detection as measured by levels of luminescence for a panel of different types of compounds for mouse odorant receptor (mOR9-I). Based on the range of different compounds tested, mOR9-1 may be more narrowly tuned to one (vanillic acid) compound or a smaller subset of ligands. A narrowly tuned receptor, may help detect a specific compound or a specific smaller subset of compounds.

### Signal Detection

Devices as described herein may include one or more isolated cells (such as a neuron). The isolated cell may be utilized as a sensing front-end to detect specific volatile organic compounds from or within an environment. An isolated cell may sense a presence of a compound in real-time in an environment, or from an isolated sample (air sample, liquid sample, solid sample) that was previously taken from an environment to be tested.

One or more isolated cells within the device may alert the detection system that a binding event (between a cell receptor and a compound) has occurred by creating one or more of a biological signal, a light signal, a chemical signal, an electrical signal, a vibration signal, a mechanical signal, or any combination thereof In the case of a neuron, the signal may comprise firing an electrical impulse, such as an action potential or a portion of an action potential. A signal emitted from an isolated cell of the device may be detected by another component of the device, such as a sensor. In the case of an electrical signal, such as an action potential, one or more electrodes (such as a gold electrode) may receive this electrical signal. Electrodes of an device may sense one or more ionic fluxes including one or more of Na+, Ca2+, K+ luxes, or combination thereof. These ionic fluxes may pass in and out of the membrane of the isolated cell to generate one or more electrical signals, and the resulting electron flow may be translated to one or more electrodes, which may then be translated by an analogue to digital converter to a waveform which may then be classified by downstream algorithms as either an electrical signal (i.e., action potential or portion thereof) or noise.

The difference between a noise signal and an electrical signal (i.e., action potentials or portion thereof) may be termed the signal- to-noise ratio (SNR). The SNR may determine a confidence level of a detection event. The SNR may define a level of signal power with respect to a level of noise power, where noise may be a result of a physical turbulence or electromagnetic noise emanating from one or more surrounding electronics. The quality of a cellular recording (such as recording action potentials of a neuron in response to the presence of a compound) may be deternined by an amplitude of a voltage of a recorded spike. This amplitude may be increased by improving a contact interface between an isolated cell (such as a neuron) and a surface of an electrode. In some cases, increasing a contact interface may include reducing a physical distance between the isolated cell and a surface of the electrode, increasing a contact surface area between the isolated cell and the electrode, or a combination thereof Both approaches of improving contact interface may be optimized by augmenting electrode geometry to encourage the isolated cell (i.e., neuron) to engulf the electrode, such as a geometry that may advantageously trigger one or more endocytotic pathways in the isolated cell. By triggering one or more endocytotic pathways in an isolated cell, the isolated cell may begin to consume at least a portion of the electrode via partial phagocytosis, which may increase the cell membrane surface area participating in ion exchange that may be in close proximity to the electrode for an electrical current to be generated in a portion of the electrode and amplified by an amplifier and digital to analogue converter.

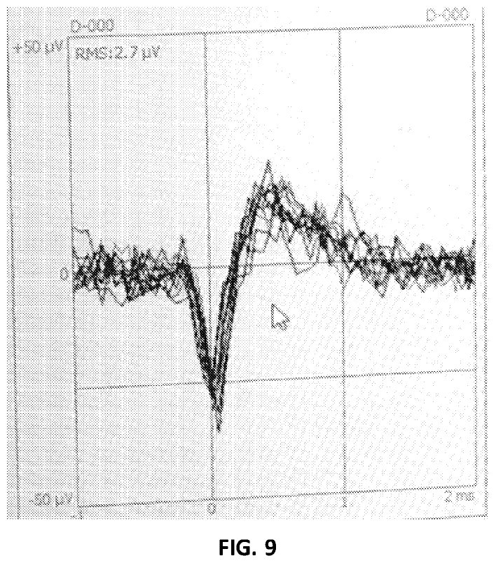

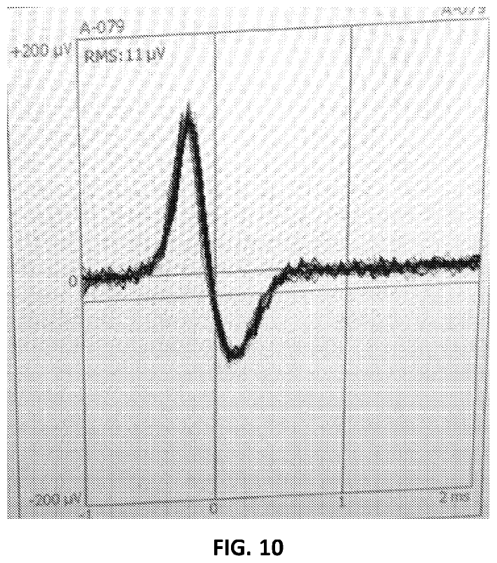

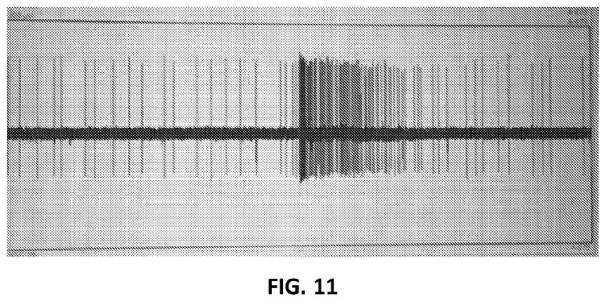

FIG. 9 shows an amplitude gain obtained employing a planar 2D electrode geometry. The image is generated by Intan recording software. The image demonstrates an average of about 50/3 uV peak-to-peak signal quality based on a distance between the maximum and minimum peaks present in the image. FIG. 10 shows a superior amplitude gain obtained employing a 3D electrode geometry showing far superior amplitude gain as compared to the planar 2D electrode geometry. This image demonstrates an average of about 200/3 uV peak-to-peak signal quality based on the distance between the maximum and minimum peaks present in the image. In addition, the signal-to-noise (SNR) ratio is significantly improved using the 3D electrode geometry in FIG. 10 as compared to the 2D electrode geometry in FIG. 9. A 3D electrode geometry may result in a fourfold signal gain nover 2D electrode geometry. A 3D electrode geometry may result in a 5, 6, 7, 8, 9 or 10 fold signal gain over 2D electrode geometry. FIG. 11 shows a spike train obtained using a 3D electrode geometry.

### Devices for Detection

The devices, systems, and methods as described herein may be employed to confirmation a presence or an absence of one or more compounds in a sample. In some embodiments, the confirmation may be a likelihood of a presence or a likelihood of an absence of one or more compounds in a sample. A sample may be a blood sample, bodily fluid, tissue sample, or any combination thereof. In such embodiments, a presence or absence of an illegal compound in a subject's body may be confirmed. A sample may be a soil sample, a water sample, or a gas sample. In such embodiments, a presence or absence of a chemical weapon, a toxin, a carcinogen in a soil, in a waterway, in an air supply, in a geographical region, in a residential setting, in a commercial setting may be confirmed.

The system may comprise an array of cells. These cells may be from any suitable origin. For example, they may be simulated, synthetic or natural. The array may be intended to provide the cells with life support, capable of maintaining a suitable environment for these cells, including temperature control and delivering nutrients and other materials to the cells. As such, the array may be combined with other systems for the transportation of living cells, without interruption to normal physiological functions. The preferred embodiments of the disclosure provide electrodes for interaction with the cells, in order to monitor signals, such as electrical signals. Preferred embodiments of the disclosure also allow delivery of compounds to the cells and monitoring the cell response. Preferred embodiments of the disclosure may also support imaging modalities to monitor the cell response or function.

In general, the array may provide a structure (which can be considered to be a substrate (in x, y, z coordinates)) in which the cells may be housed, the same structure providing an arrangement for perfusion of the cells with nutrients, growth media, growth factors, compounds, etc.

The array may be intended to form part of a base unit, providing a gas delivery system which drives the perfusion of cell culture media through the array. Additional elements of the array may include heating elements, for maintaining life support in the array. Various sensors may also be included to monitor the temperature, pH, gas species, particle analysis, etc., in closed loop.

In addition to the foregoing, other sensors may be provided, e.g. adapted to sense the presence of specific proteins or ionic molecules. Such sensors and detectors can interface with the array in a secondary manner to provide analytic read outs for genomics, proteomics, western blot assays and other lab-on-chip devices.

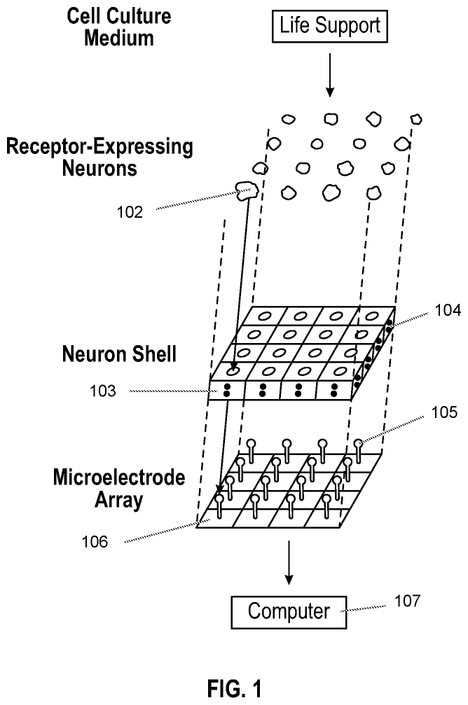

In FIG. 1, shows a life support system 100 that may be operatively connected to the array of cells. The life support system may provide a controlled environment of a liquid volume, such as a cell culture medium 101, under a controlled pH, a controlled temperature, a controlled osmolality, or combinations thereof Compounds of interest may be dissolved into the liquid volume. One or more compounds added to the liquid volume may interact or bind to one or more receptors of receptor- expressing neurons 102 of the array which may trigger a signal, such as an electrical signal. The neurons may reside in a neuron shell 103. The neurons may be individually placed through an opening at the top of each neuron shell, represented by a small circle 104 in FIG. 1. Each neuron shell may be positioned about a subset of microelectrodes represented by a single electrode 105 in each square of the grid 106. One or more electrical signals may be collected from the neurons excited by one or more compounds. The one or more electrical signals measured by the electrodes may be received by a controller, such as a computer 107 as represented by the black arrow at the bottom.

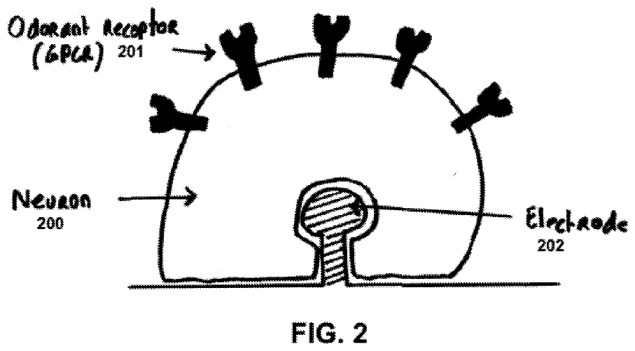

In FIG. 2, a neuron 200 expressing more than one odorant-receptor 201 may be placed atop a subset of electrodes (such as gold microelectrodes) represented in FIG. 2 by one electrode 202. Compounds of interest may bind to the odorant- receptor 201 leading to a cascade of events in the cell cytoplasm of the neuron 200 which may lead to an electrical signal, such as a membrane depolarization and possibly an action potential or a train of action potentials. The electrical signal may be measured or recorded by the electrode 202.

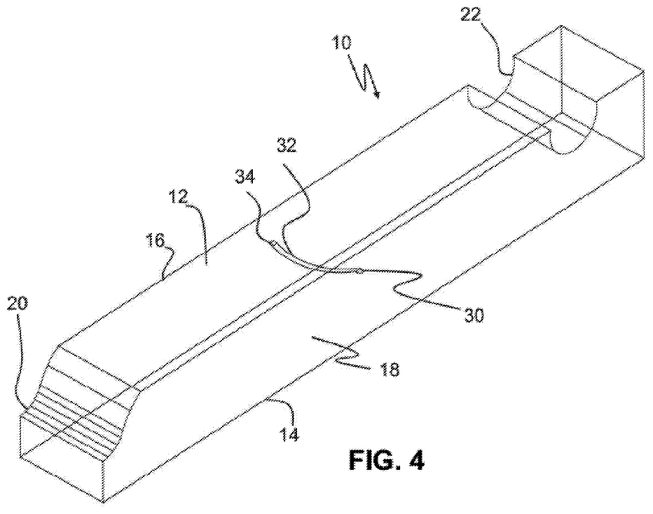

In FIG. 4, an array may be generally indicated by reference number 10. This may have a generally elongate prismatic shape with an upper region bounded by upper surface 12 and a lower region bounded by lower surface 14. Upright side surfaces 16, 18 may also be provided.

Front region 20 may be provided with a profile surface, for integration of the array into the system. Additionally, a cut-out 22 may be formed towards the rear part of the upper region of the array. The front and rear shapes of the array may provide purchase for a hooking system to be able to pick up the array and place it in a base unit (not shown).

In FIG. 4, chamber 30 is shown, located in the lower region of the module.

Chamber 30 may house one or more cells (not shown). Chamber 30 may have a respective cell introduction passage 32, leading from cell introduction port 34 at upper surface 12 of the module. As can be seen in FIG. 1, the cell introduction passage may slope and curve generally downwardly from the upper region to the chamber in the lower region. This feature may permit one or more cells to be introduced into chamber 30 via port 34 and passage 32, and may provide certainty as to the number and type of cells contained in chamber 30.

The system may comprise an array of cells, such as neurons, that may be modified, such as genetically modified, to express cell-surface receptors. The cell-surface receptors may detect one or more compounds such as a volatile organic compound or a water-soluble odorant compound. A cell of the system may express a single type of compound- sensing receptors or may express multiple types of compound-sensing receptors that may detect a set or a mixture of compounds. The array of cells may comprise one or more cells expressing one or more of these compound-sensing receptors. A cell-surface receptor that may sense a compound may do so via a series of signaling proteins that internally amplify a signal, such as an electrical signal, and may trigger an action potential by the cell, such as a neuron. Each individual cell may be operatively connected to one or more electrodes of the system, such as the microelectrode array (MEA). Following or during a binding event between a compound and a cell-surface receptor, the operative connection between a cell and an electrode may permit detection of one or more cell-based electrical signals, such as a cell membrane depolarization, an action potential, or an electrical signal that is subthreshold of an action potential. An electrical signal response may be transferred to an electrode and to a controller, such as a computer input device. In some embodiments, the array of cells differentially detects an array of compounds, which collectively can yield a compound fingerprint of detection.

A compound (such as an odorant compound) binding to cell-surface receptor (such as an odorant receptor) in a cell (such as a neuron) expressing one or more of that type of cell-surface receptor may active a signaling pathway within the cell. In some embodiments, wherein the cell is a neuron, a binding event may trigger a membrane depolarization of the neuron and in some cases also trigger an action potential, both of which are electrical signals that can be detected by an electrode that may be operatively connected to the cell. In some embodiments, a complete action potential may not be triggered. In such cases, an electrode may still detect an electrical signal from the cell, such as detection of a sub-threshold level signal that may be distinguishable from noise. An excited neuron may generate one or more action potentials to a given binding effect, the one or more action potentials of which may be grouped, stored, or analyzed as a train of action potentials by a controller. A distribution pattern of action potentials within a train of action potentials may also contain electrical signal information which may be collected by the system.

In some embodiments, an array of cells may comprise a plurality of cells, wherein each cell of the plurality expresses a unique cell-surface receptor. In some cases, a compound, such as an odorant, may bind differentially across the plurality of cells such that each cell may be have a different electrical signal level between a baseline line, such as no action potential (i.e. zero), and a lull action potential. In some embodiments, different cells may generate a train of action potentials that may have different or unique action potential distributions within the train, depending on the cell-surface receptor that may be expressed on the cell surface.

Delivery of a single compound (such as an odorant) or a set of compounds with known characteristics to an array may provide a series of relative signals across the array that can be obtained and analyzed by the devices, systems, and methods as described herein. Values associated with the relative signals may be imported or contained within a matrix containing different levels (such as a magnitude) and profiles (such as a temporal profile) of electrical signals for each cell, based on sub-threshold signals, full-threshold signals, membrane depolarizations, action potentials, or any combination thereof

A single compound may bind to different cell-surface receptors with different binding affinities. A single compound may have a strong binding affinity to one type of cell-surface receptor and a weak binding affinity for a second type of cell-surface receptor. Different binding affinities, such as a strong binding affinity and a weak affinity, may result because a binding to a G protein coupled receptor (GPCR) is a 3-dimentional binding event. In some embodiments, binding sites of cell-surface receptors may not be sensitive enough to recognize particular moieties or chemical substituents (e.g., OH, CH3, etc.) that comprise a given compound of interest. Instead, it may be that a combination of features of the compound may be sufficient to provide a ligand “shape” or a conformation that permit a binding event to occur between the compound and the cell-surface receptor inside the GPCR binding pocket. Thus, different parts of a compound may bind to different cell-surface receptors differently and may trigger different signals in different cells on the array. In some embodiments, a cell that binds to a compound with a strong binding affinity may provide a signal in response to that binding event that is different that an electrical signal produces by a cell that may have a weak binding affinity for the same compound. In some embodiments, cell-surface receptor binding sites may be sensitive enough to recognize particular moieties or chemical substituents (e.g., OH, CH3, etc.) that comprise a compound of interest.

A compound may bind different receptors with different binding affinities. For example, a narrowly tuned receptor (such as a modified receptor that may be modified to bind to a specific compound with a strong binding affinity) may only bind to a limited subset of compounds and a binding event to a compound to which the receptor is not narrowly tuned may not occur or may be less likely to occur. Broadly tuned receptors (such as a modified receptor that may be modified to bind to a wider range of compounds with a strong binding affinity) may bind to a larger number of different compounds with different binding affinities as compared to the narrowly tuned receptor. Broadly tuned receptors may be more likely to be useful in a chemical fingerprint determination. Broadly tuned receptors may be more likely to provide a confirmation of a presence of a compound in a sample with a high probability or degree of confidence, such as 70%, 75%, 80%, 85%, 90%, 95%, 96%, 97%, 98%, 99% or more.

A given compound may have a relatively fixed set of values in a matrix, with a range of variation across all values (such as non-zero values). For example, a first exposure of the given compound to an array of cells, may yield a first set of values in a matrix. A second exposure of the given compound to the same array of cells, may yield a second set of values in the matrix. The first set of values and the second set of values may be relatively the same set of values. The first set of values and the second set of values may have a range of variation across all values, wherein the variation may be not more than 1%, 2%, 3%, 4%, 5%, 6%, 7%, 8%, 9%, 10%), 15%). This relatively fixed set of values of the matrix may be used as a fingerprint or a unique pattern that may be assigned to that particular compound (and may be stored in a database of the system) given the distribution of the cell-surface receptor expressing cells.

A set of compounds (related or unrelated to one another) may have a particular fingerprint when mapped against a particular set of cell-surface receptors expressed by cells of an array. This fingerprint for a given set of compounds may represent an overlapping set of binding events of individual compounds. That is, individual compounds in the set of compounds may bind to more than one cell-surface receptor in different ways. In such a case, a given set of compounds may be additive across the array and the signals from some compounds in the given set may mask or render undetectable the individual signals from other compounds in the set. Each set or combination of compounds may have a unique fingerprint across the array. A pattern of signals generated by a given compound within a mixture of compounds on an array may act as a compound fingerprint which can be recognized within a mixture of compounds thereby allowing a system as described herein to determine individual compounds in a set or mixture of compounds.

The systems described herein provide an array of cells, such as neurons, having receptors which bind to an odorant or a compound, such as a volatile organic compound, and upon binding, the cells send detection information, such as signals (such as electrical signals), to a controller.

### Computer Systems

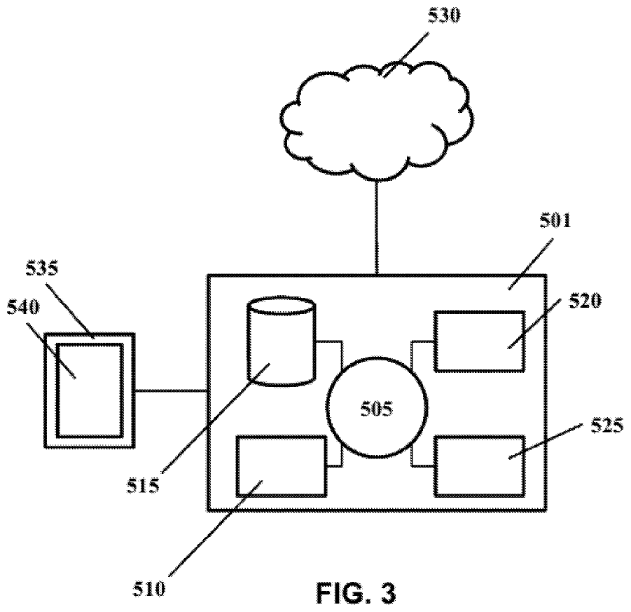

The present disclosure provides computer control systems that are programmed to implement methods of the disclosure. FIG. 3 shows a computer system 501 that may be programmed or otherwise configured to direct electrodes to measure one or more electrical signals, to receive one or more electrical signals from one or more electrodes, to generate a pattern of electrical signals, to store patterns of electrical signals or electrical signals in a database, to compare a pattern of electrical signals to a pattern stored in a database, or any combination thereof The computer system 501 can regulate various aspects of data collection, data analysis, and data storage, of the present disclosure, such as, for example, directing electrical signal measurements, comparing of patterns based of electrical signals measured, generating patterns based on electrical signal data, any combinations thereof, and others. The computer system 501 can be an electronic device of a user or a computer system that is remotely located with respect to the electronic device. The electronic device can be a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., portable PC, tablet PC, Smart phones).

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine- executable instructions are stored on memory 510.

Specific Embodiments

A number of devices, systems, arrays, methods are disclosed herein. Specific exemplary embodiments of these devices, systems, arrays, methods are disclosed below.

Embodiment 1. A device comprising: (a) a spatially addressable array, the spatially addressable array comprising: a plurality of chambers, wherein each chamber of the plurality of chambers comprises: (i) a cell modified to express a unique odorant receptor profile; and (ii) an electrical component configured to measure an electrical signal in the cell, (b) a controller configured to (i) receive the measured electrical signals from the plurality of chambers and (ii) determine a presence or an absence of one or more compounds based on the measured electrical signals.

Embodiment 2. The device of embodiment 1, wherein the presence or the absence of the one or more compounds comprises measuring an amount of the one or more compounds.

Embodiment 3. The device of embodiment 1 or 2, wherein the cell comprises a genetic modification.

Embodiment 4. The device of any one of embodiments 1-3, wherein the cell is a neuron.

Embodiment 5. The device of any one of embodiments 1-4, wherein the cell is a human cell.

Embodiment 6. The device of any one of embodiments 1-5, wherein the spatially addressable array comprises greater than three unique odorant receptor profiles.

Embodiment 7. The device of any one of embodiments 1-6, wherein the device is configured to detect at least two different types of compounds.

Embodiment 8. The device of any one of embodiments 1-7, wherein the cell detects more than one type of compound.

Embodiment 9. The device of any one of embodiments 1-8, wherein the cell is modified to express at least two unique odorant receptor profiles.

### EXAMPLE 1

Although neurons expressing different olfactory receptors can act as primary sensors for odor recognition when coupled to electrodes, the signatures of the different compounds may be embedded into complex spatiotemporal patterns of electrical activity across the different electrodes present in the system. This may be the result of different neurons reacting with different affinities to the compounds present in the air around them, as well as the fact that each electrode may be in electrical proximity to multiple neurons, and accumulating the signals from each of them.

The mapping from spike patterns to odor detection is challenging for several reasons including one or more of the following:

a. A single neuron may express different types of receptors.

b. Different neurons may express a receptor in different quantity.

c. Receptors may be broadly tuned (such as they can respond equally well to different chemicals).

d. Cells may be in communication with one another—such as neurons that may be connected via excitatory or inhibitory synapses to other neurons, causing a secondary unpredictable response to one or more odorants in the network.

e. Smells, environments, or other sources for detection may be composed of

hundreds of different molecules.

Although the consequences of many of the above points may be avoided or limited with appropriate experimental consideration, even in the worst case scenario, different odorants may be classified from simulated data through a supervised learning algorithm that may receive as input the raw neuronal activity and may output the most likely compound or smell that elicited it with sufficient accuracy. In fact, some of the above points may be exploited to improve detection robustness overall (such as sensitivity or specificity), for example, the ability of broadly tuned receptors to respond to different compounds to different degrees.

This demonstration to classify different odorants is done in 3 parts:

a. Realistic simulation of electrophysiological properties of olfactory neurons.

b. Simulation of realistic olfactory responses of neural networks expressing a

mixture of olfactory receptors.

c. Training of ML algorithm to discriminate between different patterns of evoked electrical activity.

Realistic simulation of electrophysiological properties of olfactory neurons.

To understand this argument from a bottom up approach, it may be important to examine and understand the internal cellular cascade that generates action potentials as a result of odorant binding, especially how odors binding to different receptors, or receptors with broad affinity for many environmental stimuli, can affect the spiking behavior of these cells and networks. Such broad affinity receptors may have the potential to cause false positive detection results, however, this section will demonstrate through single cell computational models that even broadly tuned receptors can modify spiking behavior in a distinguishable manner.

In the task of identifying a particular odorant using a neural-cell based biosensor, it may be important to address the problem of false positives. Due to the nature of receptor biology, some receptors may be broadly or narrowly tuned to different classes of ligands. That is, while some receptors may have extremely specific interactions with only one ligand, and bind to only one particular chemical, many receptors may be more broadly tuned. These broadly tuned receptors may respond very strongly to one ligand, but they may also have weaker interactions with similar, but altogether different, molecules. Because of this phenomenon, there may be off-target interactions wherein the receptor may respond as though the compound of interest had been detected, but it may be in reality a structurally similar, but different chemical. Because the interaction of ligands with receptors may be defined by the electrostatic interaction of the molecule with the binding domain of the protein, the affinity of the molecule for the receptor may necessarily be different for a different molecular structure. How much affinity a receptor has for a certain kind of compound may be defined by the dissociation constant, Kd, which may be defined as the propensity of the protein-ligand complex to separate (dissociate) reversibly back into just the receptor and just the ligand alone.

Based on data as described herein, compounds with different affinity for a receptor of interest may result in significantly different levels of cAMP-associated luminescence in a cell-based assay. The question remains if this difference may be preserved at the level of the action potential, or spike burst, level of cellular response. A mathematical model developed by Dougherty et al. (Dougherty D P, Wright G A, Yew A C. Computational model of the cAMP-mediated sensory response and calcium-dependent adaptation in vertebrate olfactory receptor neurons. Proc Natl Acad Sci USA. 2005;102(30):10415-20) has captured the entire signal transduction cascade from ligand docking, through G protein activation and the eventual generation of currents from cyclic nucleotide- gated channels which eventually may result in action potentials, a detection readout of devices as described herein. This model may capture the kinds of adaptation and desensitization behaviors that may have been observed in patch-clamp recordings of neurons exposed to varying concentrations of odorants, so it may be a reliable proxy for the internal cellular pathways that determine how a cell responds to odorant stimulation. The question may be, does a lower, or off-target, binding event (such as a false positive) result in a noticeable change in the responsive spike pattern, and if so, may these two spike trains be reliably discriminated? Data described herein may show that not only can this complex be modeled but also the response resulting from ligand s can be classified with greater or lesser affinity for the receptor algorithmically.

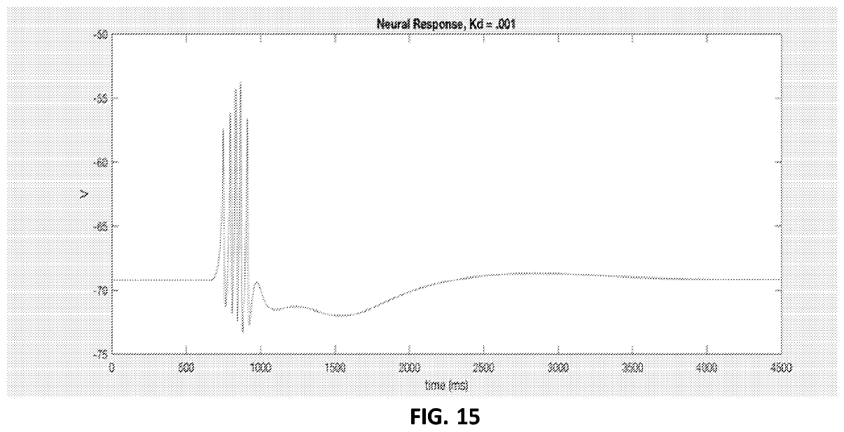

The trace in FIG. 15 represents the spiking behavior of a model neuron expressing a model odorant receptor with a low defined dissociation constant for a ligand exposed to the cell for a 50 millisecond (ms) pulse. A low dissociation constant, as mentioned, means that the ligand may have a high affinity for the receptor and may activate it and the related downstream signaling cascade for a comparatively long time. Due to the low dissociation constant, a substantial spike burst with a 500 ms total duration results.

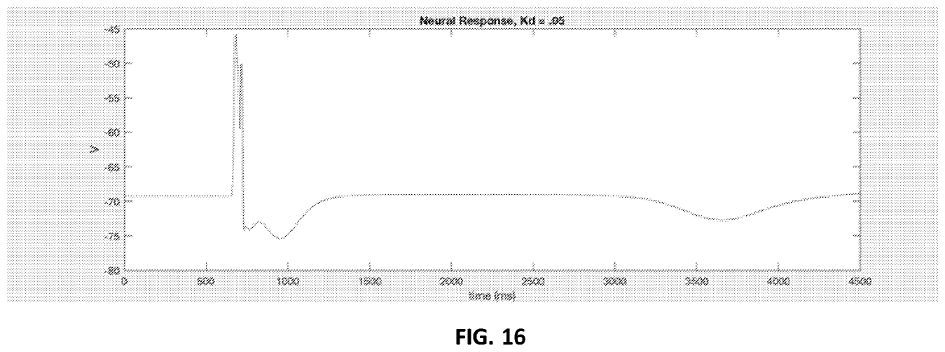

Conversely, the trace in FIG. 16 results from the exact same model neuron, but exposed to a 50 ms pulse of a ligand for which the receptor may have a high defined dissociation coefficient, meaning that the ligand may be likely to separate from the receptor often, resulting in less activation of the downstream signaling cascade. This results in fewer action potentials and a shorter spike burst of only around 100 ms.

Based on these simulation results, there may be clear differences between the spiking behavior resulting from ligand-receptor complexes of different affinities. Therefore, even on a single cell level, off-target detection events characterized by ligand-receptor interactions with higher dissociation coefficients may be measurably different from those narrower, on-target interactions that characterize an actual, true positive detection event.

Simulation of realistic olfactory responses of neural networks expressing a mixture of olfactory receptors.

In the event that the readout may be in actuality the net response of an entire population of cells, a different and perhaps even more robust tactic can be employed to discriminate the neural responses to different odorants. In such a population of neurons, there may be a number of different receptors expressed on the surface of each cell. If these receptors may be all broadly tuned for the odorant in question, they may each engender a different level of response to the same chemical stimulus. For example, receptor A, expressed in one cell type, may bind the odorant with 50% affinity, receptor B with 30% affinity, and receptor C with 80%>affinity. This pattern can be used to uniquely identify the compound from the spiking activity, because as demonstrated herein, different ligand-receptor affinities may result in different spiking behavior in a cell expressing that receptor. In some cases, a similar compound, with a similar chemical structure, which may generate an off-target response in these receptors, may activate each receptor at a different level (receptor A with 20% affinity, receptor B with 80%) affinity, and receptor C with 80%>affinity), therefore producing a completely different activity pattern, and it may therefore be distinguished from the actual chemical of interest by examining the network behavior overall.

From a more global perspective, in an interconnected network of neurons, each cell may express the odorant receptors of interest at different levels, with some cells having very high levels of expression and many cells no expression at all. For this reason, it may be important to examine if and how the few neurons with a high enough level of expression of the correct receptor to respond to the chemical of interest may impact the overall network activity. For example, if the remainder of the neurons respond unpredictably and for extended intervals to a provoked odor stimulus, then detection events may become difficult. The computational simulation below demonstrates that the few responsive neurons may not unpredictably perturb the overall network activity to a degree that would make proper odorant identification and classification unfeasible.

To see the impact of “odorant- stimulated” neurons at the network scale, a network of 500 lzhikevich spiking neurons [lzhikevich (2003)] were created in Python. The lzhikevich model is a set of coupled differential equations which model biophysically realistic spiking neural behavior. The neurons connect to one another via randomly distributed variable weight synapses. Additionally, a Gaussian noise was introduced to a random fraction of the network for the duration of the experiment. This simulates random odorant-associated stimulation of the small fraction of the network which is transfected with a receptor at an appropriately high level to generate spiking behavior. To interpret experiments, raster plots are used, with the time step in the x-axes and the neuron number that has fired in y-axes.

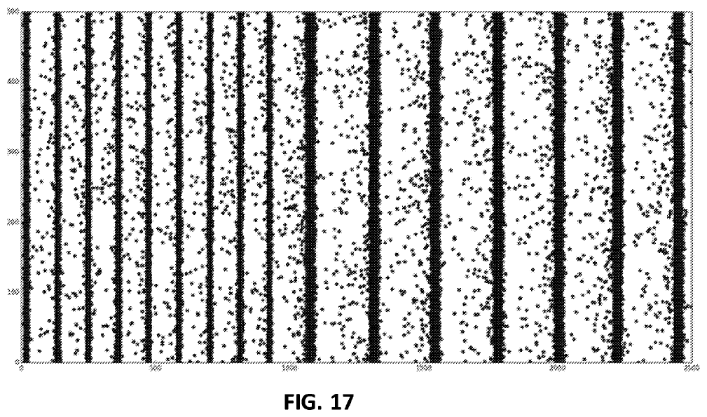

As shown in FIG. 17, a baseline experiment is first run to see the impact of the gaussian noise introduced to the random fraction of the network. Interestingly, the frequency of burst may be reduced at mid- simulation. This may be probably due to the intrinsic properties of the synapses as coded into the model.

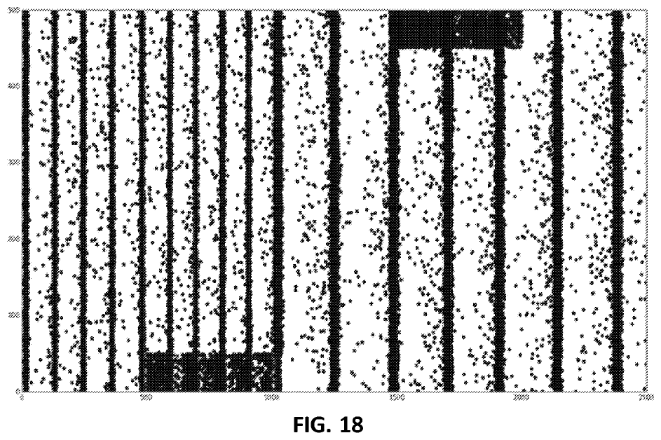

Experiment 1 as shown in FIG. 18, demonstrates that even with a certain level of background noise, a signal for each of the two narrowly tuned receptors is distinguishable. The first experiment utilized 2500 time steps for solving the associated model spiking equations. The network is comprised of two subpopulations of neurons with the following characteristics: (a) Subpopulation 1 has an affinity of 100% for odorant A and 0% for odorant B; (b) Subpopulation 2 has an affinity of 0% for odorant A and 100% for odorant B. (Note: on the raster plot of FIG. 18, Subpopulation 1 is the 50 first neurons at the bottom of the plot, and Subpopulation 2 is the last 50 neurons at the top of the plot). In the raster plot of FIG. 18, the following time course of stimulation was applied to the network (time on the x axis): 0-500: No odorant; 500-1000: release of odorant A; 1000-1500: No odorant; 1500-2000: release of odorant B; 2000-2500: No odorant.

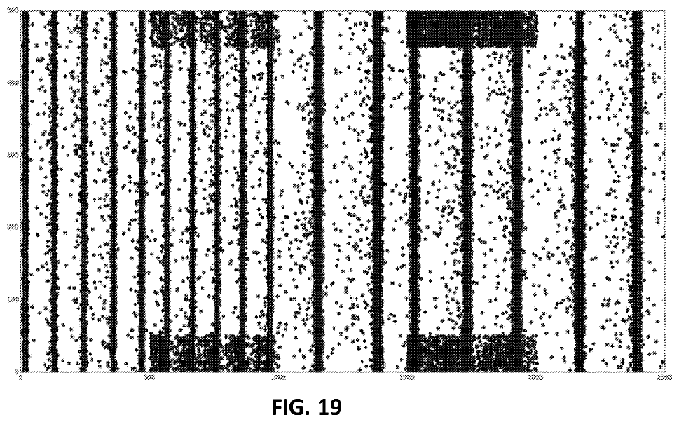

Experiment 2 as shown in FIG. 19 demonstrates that even with two different broadly tuned receptors, the two can still be distinguished based on a measured electrical output (wherein the distinguishable different may be based on a difference in density of a given region on a raster plot). The second experiment also utilized 2500 time steps, with two subpopulations of neurons that have the following characteristics: (a) Subpopulation 1 has an affinity of 70% for odorant A and 30% for odorant B; (b) Subpopulation 2 has an affinity of 30% for odorant A and 70%) for odorant B. In the raster plot of FIG. 19, the following time course of stimulation was applied to the network (time on the x axis): 0-500: No odorant; 500-1000: release of odorant A; 1000-1500: No odorant; 1500-2000: release of odorant B; 2000-2500: No odorant.

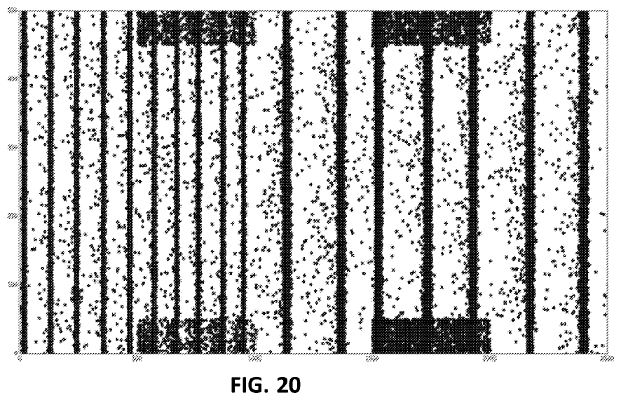

In the raster plot of FIG. 20, the following time course of stimulation was applied to the network (time on the x axis): 0-500: No odorant; 500-1000: release of odorant B; 1000-1500: No odorant; 1500-2000: release of odorant A; 2000-2500: No odorant.

In spite of the connectivity within the network, only the neurons that are directly stimulated may significantly increase their firing frequency. Indeed, these experiments may demonstrate that at a large time scale, the increase in burst frequency and duration in the rest of the network upon stimulation may be small as compared to baseline. This may mean that the recorded evoked activity may be necessarily a result of neurons directly responding to the odorant in question, not random extraneous network perturbations which may result in a potential false positive. In some cases, classification of a stimulus may occur without direct access to the signal of the neurons that may be directly responding. This situation may arise if the cultured neurons transfected with the appropriate odorant receptor are physically distant from the recording electrodes.

These simulations may demonstrate that in networks of subpopulations of neurons randomly modified, as the case would may be in a “batch” genetically modified neural network, with receptors with varying degrees of affinity for two different odorants, can respond in a distinguishable manner to a time course of stimulation with the two odorants in question, showing that even in a worst-case of uncontrolled levels of receptor expression and off-target affinity, similar odorants can still be reliably discriminated.

### EXAMPLE 2

Training a machine learning (ML) algorithm to discriminate between different patterns of evoked electrical activity.

As described herein, for example in Example 1, single neuron and network electrical activity may differ as a result of exposure to different compounds in the environment. Classifiers may be developed to reliably distinguish these different detection cases and to discriminate the true positive results from the false positives generated by structurally similar chemicals which may result in the aforementioned off-target partial receptor activation.

Different experiments that may correspond to different real life situation may be considered, for example, (a) neurons that may be directly responding to an odorant, or (b) only neurons that may be responding as a result of their synaptic connections to directly stimulated neurons. The two cases may be distinguished, (i) one case where the neurons may respond very specifically to either compound, the (ii) other case where they only have a moderate bias towards one or the other analyte—because of overlapping expression, or because of broadly tuned receptors.

Protocol (for Experiments 1, 2, 3 as described below):

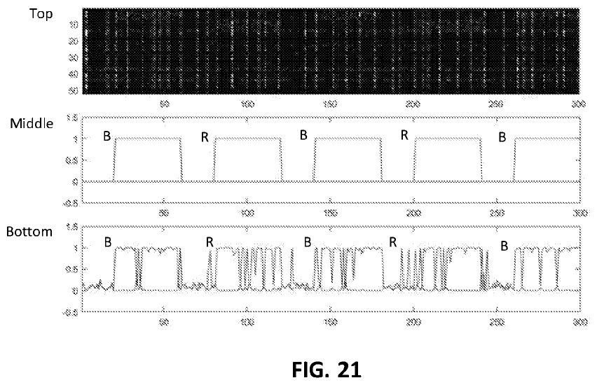

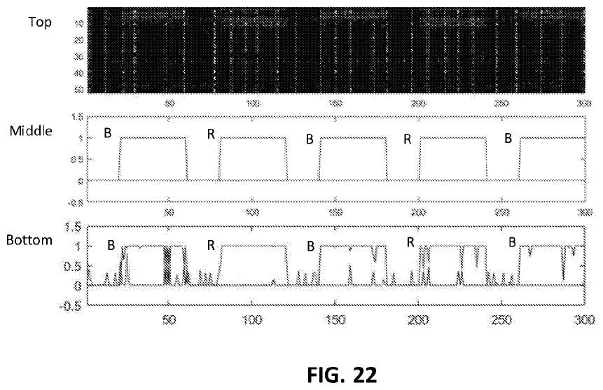

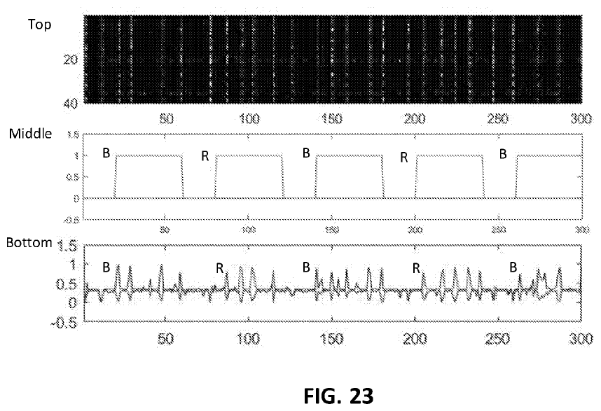

A small population of 50 neurons is generated, 2 subpopulations of 5 neurons each (10% of the total size of the network) are modified each to respond preferentially to chemical A or to chemical B. The network is simulated, and the number of spikes for each neuron is summed using a bin size of 50 ms (see top panels of FIG. 21, FIG. 22, and FIG. 23). Periodically, chemical A, chemical B, or no chemical are introduced, causing the subpopulations to respond accordingly to the receptor they express (and their respective affinity for compound A or B).

Each bin is labelled with “chemical A” or “chemical B” or “nothing” accordingly to the chemical present at that time, creating a whole data set used for training and validation. The whole simulation consists in 100 trials of alternating 2 second periods of exposure to A or B, with 1 second rinse in between. The bins of the first 70 stimuli are used as training dataset for the machine learning classifier, and the last 30 for validation. This represents a total of 2/0.05×70=2800 training example for chemical “A”, and another 2800 for chemical “B”, and 1/0.05*140=2800 for “baseline”, or no chemical at all.

The data is used to train an artificial neural network with an input vector size of N×S, where N is the number of biological neurons stimulated in the culture, or just a subset of these neurons, and s is the number of consecutive time bins that the artificial neural network sees at once, 500 hidden units, and 2 output units, corresponding to A and B. Neurons in the hidden and output layer have a sigmoid activation function. Training is done using backpropagation to compute the gradient and adadelta, a gradient descent optimization algorithm, is used to update the weights.

### Results:

### Experiment 1:

The receptors are narrowly tuned (responding 100% to compound A and 0% to B) and the signals are directly recorded from the cells having the narrowly tuned receptors (i.e. the neurons responding directly are “visible” in the input data, meaning that we can directly record electrical information from the neurons transfected with the odorant receptor tuned to respond to the odor stimulus.) The x axis is time and the y axis is a probability of predicting a compound or odorant. The top panel shows raw data in a raster plot. The middle panel shows the experimental conditions or experimental truth. The bottom panel shows the predicted outcome.

As shown in FIG. 21, odorant classification from complex activity patterns in Experiment 1. Top panel: a binned raster plot showing 15 seconds of activity (x-axis) over 50 neurons (y-axis). Middle panel: Chemical stimulation of “A” marked as “B” and “B” marked as “R” from the raster plot above. The subset receives an odorant current of 10 or 0 when presented molecule A and B respectively. The opposite for the second subset. Bottom panel: prediction of the classifier after 2000 training epochs, produced from the data presented above. Marked as “B” is the probability that chemical A is present. Marked as “R” is the probability that B is present. The prediction matches the ground truth with good accuracy, which can be seen by comparing the second and third subplots.

### Experiment 2:

The receptors are broadly tuned and the signals are directly recorded from the cells having the broadly tuned receptors. This experiment is designed to prove that good network odorant classification may still be possible in the case of broadly tuned receptors, i.e., the case where a receptor can respond to both chemical stimuli at different degrees. The receptors are broadly tuned (responding 75% to compound A and 25% to B), and the neurons responding directly are again “visible” in the input data. The x axis is time and the y axis is a probability of predicting a compound or odorant. The top panel shows raw data in a raster plot. The middle panel shows the experimental conditions or experimental truth. The bottom panel shows the predicted outcome.

As shown in FIG. 22, odorant classification from complex activity patterns in Experiment 2. Top panel: a binned raster plot showing 15 seconds of activity over 50 neurons. Middle panel: Chemical stimulation of “A” marked as “B” and “B” marked as “R” from the raster plot above. The first subset receives a current of 15 and 5 when presented molecule A and B respectively, to model the broadly tuned receptor effect. The opposite is true for the second subset. Bottom panel: prediction of the classifier after 2000 training epochs, produced from the data presented above. Marked as “B” is the probability that chemical A is present. Marked as “R” is the probability that B is present. The prediction matches the ground truth with good accuracy.

Experiment 3:

The receptors are broadly tuned and signaled are recorded from another cell in communication with the cell having the broadly tuned receptor (such as a secondary neuron that is contacting an electrode and in communication with the primary neuron having the broadly tuned receptor). The receptors are broadly tuned (75/25), and the neurons responding directly are “invisible” in the input data, meaning that the recorded electrical response is due to the activity of secondary neurons connected to the responsive neurons synaptically. The x axis is time and the y axis is a probability of predicting a compound or odorant. The top panel shows raw data in a raster plot. The middle panel shows the experimental conditions or experimental truth. The bottom panel shows the predicted outcome.

As shown in FIG. 23, odorant classification from complex activity patterns in Experiment 3. Top panel: a binned raster plot showing 15 seconds of activity over 50 neurons. Middle panel: Chemical stimulation of “A” marked as “B” and “B” marked as “R” from the raster plot above. The first subset receives a current of 15 and 5 when presented molecule A and B respectively, to model the broadly tuned receptor effect. The opposite is true for the second subset. Bottom panel: prediction of the classifier after 2000 training epochs, produced from the data presented above. Marked as “B” is the probability that chemical A is present. Marked as “R” is the probability that B is present. The prediction matches the ground truth with good accuracy.

### Discussion:

As described herein, it may be demonstrated that for a broadly tuned receptor with a varying affinity for two different odorants, A and B, this difference in ligand-receptor dissociation may directly appear in the downstream action potential readout, therefore making odorant discrimination possible at the single-cell level based on a computer simulation modeling the entire olfactory signaling cascade, as shown in FIG. 15 and FIG. 16, wherein each figures demonstrates a detectable and distinguishable signal output.

In Example 1, it may be demonstrated that in a randomly transfected network of neurons on a chip, the neurons transfected with the ability to detect an odor, A or B, may respond far above the network baseline, making detection events possible as confounding aberrant noise from the rest of the network may be negligible, as shown in FIG. 17-20.

In Example 2, the efficacy of a machine learning classifier, in this case an ANN, may be demonstrated in discriminating between two odorants presented to the network. As shown in FIG. 21, when receptors are narrowly tuned and the neurons responding directly are part of the recording, a modest ANN may be capable of quickly learning to discriminate between two odorants, even though there may be no striking difference in global activity in the raster plot. As shown in FIG. 22, even with broadly tuned receptor preferentially responding 75% for chemical A and 25% for chemical B, and vice-versa, the network can still learn to classify between the two odorants. This is a surprising an unexpected result. The 75-25 ratio may be represented in the model by a current injected upon stimulation with either odorant and not the firing rate.

Further, as shown in FIG. 23, even if the receptors are broadly tuned and even if lacking in the ability to directly record from the neurons that are responding directly to odorant stimulations, (for instance if the electrodes are not directly contacting the modified neurons), the activity that propagates in the rest of the network may be sufficient to discriminate between the two odorants. This is also a surprising and unexpected result. In some cases, learning of the ANN may take longer (because the features to be extracted may be less obvious, as intuitively understood from the raster plot).

Advantages of arrays, devices, and systems as described herein may include (a) the ability to discriminate the presence of two or more compounds using electrical signals from secondary cells that are in communication with primary cells having the odorant receptors that can bind to the two or more compounds, (b) the ability of a device having two or more broadly tuned receptors to individually detect a presence or an absence of two or more compounds, or (c) a combination thereof

### EXAMPLE 3

An array will be configured with 3 different cells, each cell of which will express a unique odorant receptor. The array will be capable of detecting greater than 3 different compounds, each at a confidence level greater than about 85%.

### EXAMPLE 4

An environment will be diagnosed for the presence or absence of a panel of volatile organic compounds. An array will be placed into the environment. The array will detect the presence or the absence of each of the volatile organic compounds at an accuracy of at least about 85% and with a detection threshold of 10 pM for each of the volatile organic compounds.

### EXAMPLE 5

An array will be configured with nine chambers. The first three chambers (aOO, aOI, a02) will each comprises a cell having a broadly tuned receptor for DNT. The second three chambers (alO, aI I, aI2) will each comprise a cell having a narrowly tuned receptor for DNT.

The last three chambers (a20, a21, a22) will each comprise a cell having a moderately tuned receptor for DNT. Each cell will contact an electrode. The array will be placed into an environment in which DNT will be present. The cells in the second three chambers will produce an electrical signal comprising a full action potential in response to the DNT and given a value of 1.0. The cells in the last three chambers will produce an electrical signal comprising a subthreshold membrane depolarization in response to the DNT and given a value of 0.5. The cells in the first three chambers will produce an electrical signal comprising a subthreshold membrane depolarization in response to the DNT and given a value of 0.1. A matrix of electrical signals will be formulated as follows:

aOO, aOI, a02 0.1, 0.1, 0.1

aIO, aI I, aI2=1.0, 1.0, 1.0

a20, a21, a22 0.5, 0.5, 0.5

The matrix of signals will be a uniquely identifying fingerprint for that panel of receptors detecting that sample comprising DNT.

### EXAMPLE 6

An array will be configured with nine chambers. The first three chambers will each comprise a cell having a broadly tuned receptor for DNT. The second three chambers will each comprise a cell having a narrowly tuned receptor for DNT. The last three chambers will each comprise a cell having a moderately tuned receptor for DNT. Each cell will contact an electrode. The array will be placed into an environment in which DNT will be present. A second array having nine chambers will also be placed into the environment in which DNT will be present. All nine chambers of the second array will comprise a cell having the broadly tuned receptor for DNT. An electrical pattern of signaling will be measured in each chamber of both arrays. The sensitivity of detection of the DNT in the array having the broadly, moderately, and narrowly tuned receptors will be 20% greater than the sensitivity of detection of the DNT in the array having only broadly tuned receptors.

### EXAMPLE 7

An array will be configured with 30 chambers. The first 10 chambers will each comprise a cell having a broadly tuned receptor for vanillic acid. The second 10 chambers will each comprise a cell having a narrowly tuned receptor for vanillic acid (such as mOR9-I). The last 10 chambers will each comprise a cell having a moderately tuned receptor for vanillic acid. Each cell will contact an electrode. A sample comprising 10 pM of vanillic acid will be placed into each chamber of the array. A second array having 30 chambers will also receive the sample comprising vanillic acid in each chamber of the second array All 30 chambers of the second array will comprise a cell having the broadly tuned receptor for vanillic acid. An electrical pattern of signaling will be measured in each chamber of both arrays. The array having the broadly, moderately, and narrowly tuned receptors will be able to detect the presence of the IOρM vanillic acid sample as compared to the array having only the broadly tuned receptors which will not be able to detect the 10 pM of vanillic acid.

### EXAMPLE 8

An array will be configured with 6 chambers. The first 3 chambers will comprise a cell having a receptor broadly tuned to cocaine (such as mOR18-I) and narrowly tuned to heroine. The second 3 chambers will comprise a cell having a receptor broadly tuned to heroine and narrowly tuned to cocaine. When sample A having heroine is contacted to the chambers of the array a first unique pattern of electrical signals will be produced. When sample B having cocaine is contacted to the chambers of the array a second unique pattern of electrical signals will be produced. When sample C having both cocaine and heroin is contacted to the chambers of the array a third unique pattern of electrical signals will be produced. Each of the 1^{st}, 2^{nd}, and 3^{rd} unique patterns will be distinguishable from one another.

### EXAMPLE 9

The micro-electrode array (MEA) for detecting a range of odorants, representing a state, such as a ripeness state of a single piece of fruit or a batch of fruit.

In some embodiments, each neuronal cell may express multiple copies of a single odorant receptor. For redundancy, different cells may express multiple copies of a given compound receptor and other cells may express other compound receptors. A detection array may consist of cells where each odorant receptor may recognize one or more of the compounds.

With a sampling device possibly coupled to a non-specific resin-based air concentrator, which can collect an air sample containing some odorant compounds, from near the fruit or seedling, one can transport the air sample so that the detection device may be exposed directly or through a membrane containing liquid media. The odorants may then pass through the membrane or liquid covering to the neurons on the detection device. Upon binding to an odorant receptor, the G-protein pathway may signal inside the cell, may amplify the signal and an action potential may be triggered. Because each cell may have an associated electrode, an electrical impulse may be sent to a signal detector. Each electrode may be wired such that the binding of an odorant to a particular cell may result in a unique signal (based on location in the array) such that the controller may identify which cell has bound odorant.

This system may permit mapping back to the odorant receptor since each cell may uniquely express a single odorant receptor. Through the decoding of odorant receptors, one can obtain a pattern of receptors that have been activated. Thus, the particular set of odorants may yield a particular pattern. This pattern may likely be compound concentration-dependent to some extent.

Furthermore, because the electrodes permit sub-threshold signals, one can derive quantitative information from each cell, yielding some information about odorant concentration. By running standard control samples across the array, one may create a database of how well different compounds bind across the array. Furthermore, for each of these controls, one can perform detection based on a serial dilution curve, which may allow one to map back a concentration from an unknown sample.

That is, the pattern of binding across the array may be more than just a binary output, such as on/off, but may also capture some information about odorant concentration levels. The pattern of action potentials generated by sufficiently excited neurons may also contain information regarding concentration. Thus, one can map back from the results of a test sample and may be able to estimate the concentration of the odorant in the test sample.

In the case of multiple types of odorants binding to multiple cells, one may receive a more complex pattern or fingerprint for the particular mixture since it may also encode concentration information and relative concentration with overlapping effects.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention.

1. A device comprising:
(a) a spatially addressable array comprising a plurality of chambers, wherein each chamber comprises:
(i) a cell modified to express a unique odorant receptor profile; and
(ii) an electrode configured to measure an electrical signal in the cell, and
(b) a controller configured to (i) receive the measured electrical signals from the plurality of chambers and (ii) determine a presence or an absence of one or more compounds based on the measured electrical signals.

2. The device of claim 1 wherein the electrode directly measures electrical activity in another cell that is in communication with the cell modified to express the unique odorant receptor profile.

3. The device of claim 1 wherein the unique odorant receptor profile comprises an odorant receptor capable of binding two or more compounds.

4. The device of claim 3, wherein the device is capable of individually detecting a presence or an absence of the two or more compounds.

5. The device of claim 1, wherein the presence or the absence of the one or more compounds comprises measuring an amount of the one or more compounds.

6. The device of claim 1 wherein the cell comprises a genetic modification.

7. The device of claim 1 wherein the cell is a neuron.

8. The device of claim 1 wherein the cell is a human cell.

9. The device of claim 1 wherein the spatially addressable array comprises greater than three unique odorant receptor profiles.

10. The device of claim 1 wherein the device is configured to detect at least two different types of compounds.

11. The device of claim 1 wherein the cell detects more than one type of compound.

12. The device of claim 1 wherein the cell is modified to express at least two unique odorant receptor profiles.

13. The device of claim 1 wherein the electrode is a three-dimensional electrode.

14. The device of claim 1 wherein the electrode comprises a microelectrode array having a plurality of three-dimensional electrodes, each three-dimensional electrode projecting into only one of the chambers.

15. A method of detecting a presence or absence of one or more compounds in an environment, comprising:
(a) placing a spatially addressable array in the environment, wherein the spatially addressable array comprises a plurality of chambers, each chamber comprising: (i) a cell modified to express a unique odorant receptor profile; and (ii) an electrode configured to measure an electrical signal in the cell; and
(b) detecting the presence or the absence of the one or more compounds based the measured electrical signals from the plurality of chambers.

16. The method of claim 15 further comprising comparing a signaling pattern to one or more signaling patterns associated with one or more reference compounds.

17. The method of claim 15 wherein the spatially addressable array comprises greater than three unique odorant receptor profiles.

18. The method of claim 15 wherein the cell is modified to express at least two unique odorant receptor profiles selected from the unique odorant receptor profiles.

19. The device of claim 1 wherein the electrode is a three-dimensional electrode.
